SEQUENCE LISTING (37 sequences)

SEQ ID NO: 1 - protein, 118 aa, Artificial Sequence
  VH of M35-1 and M35-2
QVQLVQSGAEVKKPGASVKVSCKTSGYTFTNYYIYWVRQAPGQRLEWMGWIYPGNLNIKYNEKFKGRVTITADTSASTAYMELSSLRSEDTAVYYCARDDNYAWFAYWGQGTLVTVSS

SEQ ID NO: 2 - protein, 107 aa, Artificial Sequence
  VL of M35-1 and M35-2
DIQMTQSPSSLSASVGDRVTITCKASQDVSTAVAWYQQKPGKAPKLLIYWTNTRHTGVPSRFSGSGSGTDHTLTISSLQPEDFATYYCQQHYSTPFTFGQGTKLEIK

SEQ ID NO: 3 - protein, 119 aa, Artificial Sequence
  VH of M45-1
QVQLVQSGAEVKKPGASVKVSCKASGYTLTSYWMHWVRQAPGQGLEWMGEINPSQGRSNYNEKFKSRVTMTRDTSTSTVYMELSSLRSEDTAVYYCARRGVSGNYFDYWGQGTLVTVSS

SEQ ID NO: 4 - protein, 107 aa, Artificial Sequence
  VL of M45-1
DIQMTQSPSSLSASVGDRVTITCKASQDINTYLSWFQQKPGKSPKSLIYRSNILVSGVPSRFSGSGSGQDYTLTISSLQPEDFATYYCLQYDEFPYTFGGGTKLEIK

SEQ ID NO: 5 - protein, 5 aa, Artificial Sequence
  CDR1 of VH of M35-1 and M35-2
NYYIY

SEQ ID NO: 6 - protein, 17 aa, Artificial Sequence
  CDR2 of VH of M35-1 and M35-2
WIYPGNLNIKYNEKFKG

SEQ ID NO: 7 - protein, 9 aa, Artificial Sequence
  CDR3 of VH of M35-1 and M35-2
DDNYAWFAY

SEQ ID NO: 8 - protein, 11 aa, Artificial Sequence
  CDR1 of VL of M35-1 and M35-2
KASQDVSTAVA

SEQ ID NO: 9 - protein, 7 aa, Artificial Sequence
  CDR2 of VL of M35-1 and M35-2
WTNTRHT

SEQ ID NO: 10 - protein, 9 aa, Artificial Sequence
  CDR3 of VH of M35-1 and M35-2
DDNYAWFAY

SEQ ID NO: 11 - protein, 5 aa, Artificial Sequence
  CDR1 of VH of M45-1
SYWMH

SEQ ID NO: 12 - protein, 17 aa, Artificial Sequence
  CDR2 of VH of M45-1
EINPSQGRSNYNEKFKS

SEQ ID NO: 13 - protein, 10 aa, Artificial Sequence
  CDR3 of VH of M45-1
RGVSGNYFDY

SEQ ID NO: 14 - protein, 11 aa, Artificial Sequence
  CDR1 of VL of M45-1
KASQDINTYLS

SEQ ID NO: 15 - protein, 8 aa, Artificial Sequence
  CDR2 of VL of M45-1
RSNILVSG

SEQ ID NO: 16 - protein, 9 aa, Artificial Sequence
  CDR3 of VL of M45-1
LQYDEFPYT

SEQ ID NO: 17 - protein, 451 aa, Artificial Sequence
  Heavy Chain of M35-1
QVQLVQSGAEVKKPGASVKVSCKTSGYTFTNYYIYWVRQAPGQRLEWMGWIYPGNLNIKYNEKFKGRVTITADTSASTAYMELSSLRSEDTAVYYCARDDNYAWFAYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPPKSCDKTHTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, Artificial Sequence
  Light Chain of M35-1
DIQMTQSPSSLSASVGDRVTITCKASQDVSTAVAWYQQKPGKAPKLLIYWTNTRHTGVPSRFSGSGSGTDHTLTISSLQPEDFATYYCQQHYSTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 451 aa, Artificial Sequence
  Heavy Chain of M35-2
QVQLVQSGAEVKKPGASVKVSCKTSGYTFTNYYIYWVRQAPGQRLEWMGWIYPGNLNIKYNEKFKGRVTITADTSASTAYMELSSLRSEDTAVYYCARDDNYAWFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSDCKTKTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence; identical to SEQ ID NO: 18
  Light Chain of M35-2

SEQ ID NO: 21 - protein, 452 aa, Artificial Sequence
  Heavy Chain of M45-1
QVQLVQSGAEVKKPGASVKVSCKASGYTLTSYWMHWVRQAPGQGLEWMGEINPSQGRSNYNEKFKSRVTMTRDTSTSTVYMELSSLRSEDTAVYYCARRGVSGNYFDYWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  Light Chain of M45-1
DIQMTQSPSSLSASVGDRVTITCKASQDINTYLSWFQQKPGKSPKSLIYRSNILVSGVPSRFSGSGSGQDYTLTISSLQPEDFATYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 98 aa, Artificial Sequence
  CH1 of M35-1 and M45-1 (in the format of IgG1S142C)
ASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 24 - protein, 98 aa, Artificial Sequence
  CH1 of M35-2 (in the format of IgG2)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 25 - protein, 18 aa, Artificial Sequence
  Hinge Sequence of Heavy Chain of M35-1 and M45-1
EPPKSCDKTHTVECPPCP

SEQ ID NO: 26 - protein, 18 aa, Artificial Sequence
  Hinge Sequence of Heavy Chain of M35-2
EPPKSDCKTKTVECPPCP

SEQ ID NO: 27 - protein, 10 aa, Artificial Sequence
  Hinge Sequence of Heavy Chain of Other Examples
CKTHTCPPCP

SEQ ID NO: 28 - protein, 11 aa, Artificial Sequence
  Hinge Sequence of Heavy Chain of Other Examples
DKTCHTCPPCP

SEQ ID NO: 29 - protein, 12 aa, Artificial Sequence
  Hinge Sequence of Heavy Chain of Other Examples
ERKSCVECPPCP

SEQ ID NO: 30 - protein, 15 aa, Artificial Sequence
  Hinge Sequence of Heavy Chain of Other Examples
EPKSDCKTHTCPPCP

SEQ ID NO: 31 - protein, 15 aa, Artificial Sequence
  Hinge Sequence of Heavy Chain of Other Examples
EPKSDKCTHTCPPCP

SEQ ID NO: 32 - protein, 17 aa, Artificial Sequence
  Hinge Sequence of Heavy Chain of Other Examples
EPKSDCKTHTVECPPCP

SEQ ID NO: 33 - protein, 15 aa, Artificial Sequence
  Hinge Sequence of Heavy Chain of Other Examples
EPKSDCKTVECPPCP

SEQ ID NO: 34 - protein, 17 aa, Artificial Sequence
  Hinge Sequence of Heavy Chain of Other Examples
EPKSDKCTHTVECPPCP

SEQ ID NO: 35 - protein, 19 aa, Artificial Sequence
  Hinge Sequence of Heavy Chain of Other Examples
EPPPKSCDKTHTVECPPCP

SEQ ID NO: 36 - protein, 20 aa, Artificial Sequence
  Hinge Sequence of Heavy Chain of Other Examples
EPPPPKSCDKTHTVECPPCP

SEQ ID NO: 37 - protein, 18 aa, Artificial Sequence
  Hinge Sequence of Heavy Chain of Other Examples
EPPKSDCKTKTVECPPCP

(19) United States
(12) Patent Application Publication
(10) Pub. No.: US 2024/0002527 A1
(43) Pub. Date: Jan. 4, 2024

(54) ANTI-CD73 ANTIBODY, ANTIBODY-DRUG CONJUGATE, AND USE THEREOF

(71) Applicant: BLISS BIOPHARMACEUTICAL (HANGZHOU) CO., LTD., Hangzhou, Zhejiang (CN)

(72) Inventors: Bing XIA, Hangzhou, Zhejiang (CN); Cui FENG, Hangzhou, Zhejiang (CN); Lixia GU, Hangzhou, Zhejiang (CN); Chen LI, Hangzhou, Zhejiang (CN); Yuhong ZHOU, Hangzhou, Zhejiang (CN); Ziping WEI, Hangzhou, Zhejiang (CN)

(21) Appl. No.: 18/253,703

(22) PCT Filed: Nov. 19, 2021
(86) PCT No.: PCT/CN2021/131791

§ 371 Date: May 19, 2023

Related U.S. Application Data
(63) Continuation-in-part of application No. PCT/CN2020/130409, filed on Nov. 20, 2020.

(51) Int. Cl. C07K 16/28; A61K 47/68; A61P 35/00

(52) U.S. Cl. CPC C07K 16/2896; A61K 47/6849; A61K 47/6889; A61P 35/00; A61K 47/68031; C07K 2317/522; C07K 2317/53; A61K 2039/505

(57) ABSTRACT

Isolated anti-human CD73 antibody includes two heavy chains each including a hinge region comprising an amino acid sequence of that allow site-specific conjugation of cytotoxic drugs. Each heavy chain can include a human CH1 domain located upstream of and connected to the hinge region. The CH1 domain comprising a cysteine at the position of 142 according to the IMGT numbering scheme. ADCs containing the antibody conjugated with a cytotoxic drug are also provided. Pharmaceutical compositions including the antibody or the ADCs, and methods of treating cancer using the pharmaceutical compositions are also provided.

### CROSS REFERENCE TO RELATED APPLICATION

This application claims priority to International Application No. PCT/CN2020/130409 filed Nov. 20, 2020, the disclosure of which is incorporated by reference herein in its entirety.

### BACKGROUND

CD73 is Ecto-5′-nucleotidase (ecto-5′-NT), a glycophosphatidylinositol-anchored receptor expressed by populations of immune cells, stromal cells, epithelial and endothelial cells, cancer cells, and exosomes. This enzyme catalyzes the conversion of adenosine monophosphate (AMP) to adenosine. CD73 is believed to play a role in maintaining endothelial integrity^{[1]} as well as in mediating the inhibitory function of regulatory B and T lymphocytes^{[2]}.

In addition to the roles in the biology of healthy tissue, CD73 and adenosine each affect tumor biology. Extracellular adenosine accumulates in tumor and suppresses cytotoxic T cells and natural killer cells^{[3]}. A2AR is one of adenosine receptors upregulated during inflammation on effector T cells and its activation inhibits effector T cell proliferation, cytotoxic activity, and cytokine production [e.g., tumor necrosis factor-α (TNF-α), interferon-gamma (IFN-γ), interleukin-2 (IL-2)]^{[4]}. Whereas, A2AR activation on T regulatory (Treg) cells promotes Treg expansion and immunosuppressive activity, tumors exploit extracellular adenosine to protect the cancer cells. As reports showing that knockout mice lacking adenosine receptors have been shown to reject tumors more readily than normal mice and knockout mice lacking CD73 have increased antitumor immunity and show decreased carcinogenesis when compared with normal mice^{[5-7]}, further support tumors use CD73 to generate adenosine and, thereby, suppress antitumor immunity. Elevated CD73 expression occurs frequently in human cancers and often correlates with a worse prognosis observed in tumors of lung, colorectal, breast, ovarian, renal and glioblastoma. Clinically, elevated CD73 levels in the tumor tissue of several cancer types, including breast, ovarian, and colorectal cancers (CRC), are linked to poor patient survival^{[8]}, which underscores the crucial role of CD73 in tumor progression.

In recent years, antibodies and small molecular inhibitors against CD73 have made their way into preclinical and clinical trials as an attractive target for restoring antitumor immunity^{[9]}. Inhibiting CD73 (and/or A2AR) restores antitumor immunity in many preclinical studies with combination approaches showing superior efficacy. Preliminary safety profiles report BMS-986179, an anti-CD73 humanized monoclonal antibody, and its combination with nivolumab (anti-PD-1 therapy) to be well-tolerated in patients (NCT02754141)^{[10]}. Future studies in GI cancers that focus on determining if adenosine-mediated resistance to immunotherapy therapy exists at diagnosis or evolves with therapy will also be of significant benefit. Encouraging early results for BMS-986179 combined with nivolumab report clinical benefit (partial response) in one or more patients with pancreatic and prostate cancer^{[10]}, both are poorly immunogenic tumors. Preclinical studies show CD73/adenosine therapy (e.g., A2AR deletion) liberates CD8+ T cells for antitumor activity even against weakly immunogenic sarcomas^{[11]}. AMG510 (NCT03600883), a selective inhibitor for KRAS (G12C) recently showed promising antitumor effects, including increasing ICI therapy sensitivity in preclinical models^{[12]}. Its combination with CD73/adenosine receptor blockade may be a promising future approach.

ADCs are monoclonal antibodies conjugated with cytotoxic agents. They take advantage of target specificities to tumor cell-surface proteins and deliver toxic payload with high potencies to tumors with specificity and potency not achievable with traditional chemotherapies. There is no market approval ADCs targeting CD73. Yu et al. reported a first anti-CD73-ADC (Hu001-MMAE, DAR4) and demonstrated its potent cytotoxicity against CD73-high expressing tumor cells^{[13]}. The CD73-ADC also demonstrated high potencies in multiple in vivo tumor models including non-small cell lung cancer (NSCLC) and glioma tumors harboring RAS-mutations. However, preliminary toxicity/PK study in nonhuman primates showed that these DAR4 ADCs had short half-life even at the dose near the highest possible tolerable dose levels.

There is a need for anti-CD73 ADCs with improved PK profile and better tumor suppressive activity.

### SUMMARY OF THE INVENTION

In one aspect, the present disclosure provides an isolated antibody, or an antigen-binding portion thereof. In some embodiments, the antibody comprises: two heavy chains each comprising: (a1) a heavy chain hinge region comprising the amino acid sequence set forth in any of SEQ ID NOs: 25-37; and (a2) a heavy chain variable domain comprising a CDR1 region, a CDR2 region, and a CDR3 region comprising the amino acid sequences of SEQ ID NO: 5, SEQ ID NO: 6, and SEQ ID NO: 7, respectively, and two light chains each comprising: (a3) a light chain variable domain comprising a CDR1 region, a CDR2 region, and a CDR3 region comprising the amino acid sequences of SEQ ID NO: 8, SEQ ID NO: 9, and SEQ ID NO: 10, respectively. In some embodiments, the antibody comprises: two heavy chains each comprising: (b1) a heavy chain hinge region comprising the amino acid sequence set forth in any of SEQ ID NOs: 25-26; (b2) a heavy chain variable domain comprising a CDR1 region, a CDR2 region, and a CDR3 region comprising the amino acid sequences of SEQ H) NO: 11, SEQ ID NO: 12, and SEQ ID NO: 13, respectively, and two light chains each comprising: (b3) a light chain variable domain comprising a CDR1 region, a CDR2 region, and a CDR3 region comprising the amino acid sequences of SEQ ID NO: 14, SEQ ID NO: 15, and SEQ ID NO: 16, respectively. The antibody can specifically bind to human CD73 protein. In some embodiments, each of the heavy chains further comprises a human CH1 domain located upstream of and connected to the hinge region, the CH1 domain comprising a cysteine at the position of 142 according to IMGT numbering scheme.

In other embodiments, the antibody comprises: two heavy chains each comprising: (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; (b) a human CH1 domain located upstream of and connected to the hinge region, the CH1 domain comprising a cysteine at the position of 142 according to the IMGT numbering scheme, wherein the antibody specifically binds to human CD73 protein. The amino acid sequence comprised in the hinge region can be selected from the group consisting of SEQ ID NOs: 25-37, for example, SEQ ID NO: 25, or SEQ ID NO: 26. The antibody can comprise two kappa light chains each paired with one of the heavy chains.

In some embodiments, the CH1 domain of the antibody can have the same sequence as that of the CH-1 domain of a native human IgG2, IgG3, or IgG4 subclass antibody. In some embodiments, the CH1 domain of the antibody has the sequence of that of the CH1 domain of a native human IgG1 antibody with the mutation S142C. In some embodiments, each of the heavy chains further comprises an Fe domain of a native human IgG1, IgG2, IgG3, IgG4 subclass antibody downstream of and connected to the hinge region, wherein the Fc domain optionally includes one or more substitutions, for example, LALA mutations.

In some embodiments of the antibody, each of the heavy chains comprises a variable domain comprising the amino acid sequence set forth in SEQ ID NO: 1 or SEQ ID NO: 3, and wherein each of the light chains comprises a variable domain comprising the amino acid sequence set forth in SEQ ID NO: 2 or SEQ ID NO: 4.

In some embodiments of the antibody, each of the heavy chains comprises an amino acid sequence set forth in one of SEQ ID NO: 17, SEQ ID NO: 19, and SEQ ID NO: 21; and wherein each of the heavy chains comprises an amino acid sequence set forth in one of SEQ ID NO: 18, SEQ ID NO: 20; and SEQ ID NO: 22.

In specific embodiments, each of the heavy chains comprises an amino acid sequence set forth in one of SEQ ID NO: 17; and each of the light chains comprises an amino acid sequence set forth in one of SEQ ID NO: 18.

In specific embodiments, each of the heavy chains comprises an amino acid sequence set forth in one of SEQ ID NO: 19; and each of the light chains comprises an amino acid sequence set forth in one of SEQ ID NO: 20; or
in specific embodiments, each of the heavy chains comprises an amino acid sequence set forth in one of SEQ ID NO: 21; and each of the light chains comprises an amino acid sequence set forth in one of SEQ ID NO: 22.

In a further aspect, the present disclosure provides an antibody-drug conjugate (ADC) or a pharmaceutically acceptable salt thereof, which comprises the antibody as described herein conjugated to a cytotoxic drug by a chemical linker. The cytotoxic drug can be selected from the group consisting of eribulin, monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, and nanoparticles. The chemical linker comprises a portion that can be selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGₙ-Val-Cit-PAB (n=1-20), Mal-amido-PEGn-Val-Cit-PAB (n=1-20), MC-Gly-Gly-Phe-Gly, Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate. In some embodiments, wherein the cytotoxic drug is MMAE.

In a further aspect, the present disclosure provides a pharmaceutical composition comprising an isolated antibody or an antigen binding portion thereof as described herein, or an ADC of pharmaceutically acceptable salt thereof as described herein, and a pharmaceutical acceptable carrier. In a further aspect, the present disclosure provides a method of treating cancer in a human subject, comprising administering an effective amount of the pharmaceutical composition. The cancer can be a cancer that is associated with overexpression of human CD73 protein. The cancer can be selected from the group consisting of bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, glioma, melanoma, lymphoma, leukemia, myeloma, sarcoma, or virus-related cancer.

### BRIEF DESCRIPTION OF THE DRAWINGS

FIG. 1 shows schematic diagrams of the amino acid numbering system of antibodies as used in this application. (a) IMGT-based amino acid numbering scheme of human IgG1(κ). (b) the numbering scheme for mutants (for the IgG1 hinge region).

FIG. 2 shows the production and physicochemical testing results of the certain antibodies according to embodiments of the present invention. SDS-PAGE analysis of reducing (R) and non-reducing (NR) antibodies: M35-1 antibody (a), M35-2 antibody (b) and M45-1 antibody (c); purity and yields of the antibodies produced in HEK293 cells (d) SEC-HPLC analysis of purified antibodies, M35-1 antibody (e), M35-2 antibody (f) and M45-1 antibody (g).

FIG. 3 shows HIC profiles of ADCs made of M35-1, M35-2 and M45-1 antibodies. (a) M35-1-MMAE, (b) M35-2-M-MAE and (c) M45-1-MMAE.

FIG. 4 shows binding curves of anti-CD73 naked antibodies and their corresponding ADCs to CD73: (a) M35-1 antibody and M35-1-MMAE; (b) M35-2 antibody and M35-2-MM AE.

FIG. 5 shows internalization of anti-CD73 naked antibodies and their corresponding ADCs into CD73 expressing cancer cells. a: NCI-H1975 cells, b: NCI-H441 cells.

FIG. 6 shows cytotoxicity curves of M35-1-MMAE, M35-2MMAE, M45-1-MMAE to CD73-expressing cancer cells. a: U87MG cells, b: NCI-H1975 cells, c: NCI-N87 cells, d: NCI-H292 cells.

FIG. 7 shows in vivo efficacy of M35-1-MMAE and M35-2-MMAE against U87 glioma, NCI-N87 gastric cancer and NCI-H441 NSCLC xenografts in athymic nude mice.

### DETAILED DESCRIPTION

The present disclosure provides antibodies and ADCs targeting CD73 and their uses in antitumor capabilities.

In an aspect, the present disclosure provides anti-CD73 antibodies based on modification of anti-CD73 antibodies, and ADCs based on these modified anti-CD73 antibodies with two site-specific coupling loads, i.e., each modified anti-CD73 antibody molecule is linked with two cytotoxic agent payload with Drug to Antibody Ratio (“DAR”) of 2. Compared to previously reported DAR4 ADCs, DAR2 ADCs of the present invention can provide better PK profiles and provide better tumor tissue penetration and potentially better tumor suppressive activity.

In one aspect, the present invention provides an isolated antibody, or an antigen-binding portion thereof, comprising:
two heavy chains each comprising:
(a1) a heavy chain hinge region comprising the amino acid sequence set forth in any of SEQ ID NOs: 25-37 (for example, SEQ ID NO: 25 or SEQ ID NO: 26); and
(a2) a heavy chain variable domain comprising a CDR1 region, a CDR2 region, and a CDR3 region comprising the amino acid sequences of SEQ ID NO: 5, SEQ ID NO: 6, and SEQ ID NO: 7, respectively, and
two light chains each comprising:
(a3) a light chain variable domain comprising a CDR1 region, a CDR2 region, and a CDR3 region comprising the amino acid sequences of SEQ ID NO: 8, SEQ ID NO: 9, and SEQ ID NO: 10, respectively.

In another aspect, the present invention provides an isolated antibody, or an antigen-binding portion thereof, comprising:
two heavy chains each comprising:
(a1) a heavy chain hinge region comprising the amino acid sequence set forth in any of SEQ ID NOs: 25-37 (for example, SEQ ID NO: 25 or SEQ ID NO: 26); and
(a2) a heavy chain variable domain comprising a CDR1 region, a CDR2 region, and a CDR3 region comprising the amino acid sequences of SEQ ID NO: 11, SEQ ID NO: 12, and SEQ ID NO: 13, respectively, and
two light chains each comprising:
(a3) a light chain variable domain comprising a CDR1 region, a CDR2 region, and a CDR3 region comprising the amino acid sequences of SEQ ID NO: 14, SEQ ID NO: 15, and SEQ ID NO: 16, respectively.

The antibody can specifically bind to human CD73 protein. In some embodiments, the amino acid sequence comprised in the heavy chain hinge region is SEQ ID NO: 25. In some embodiments, the amino acid sequence comprised in the heavy chain hinge region is SEQ ID NO: 26.

Antibodies containing heavy chain in this format preferably form H-L inter-chain disulfide bond between C142 (or a cysteine near 142th position, according to the IMGT numbering system as further described below) of the CH1 domain with the last cysteine residue in the light chain. The cysteine residue in the hinge region upstream of the native CPPCP sequence forms a third H-H inter-chain disulfide bond. The cysteine at or near amino acid 142 in the CH1 domain could be introduced by mutation or insertion of a single amino acid in IgG1 subtype, or could come from the natural cysteine residue in the CH1 domain of IgG2, IgG3, or IgG4 subtypes. Compared with native H-L disulfide bonds in IgG1, which are between a cysteine in the hinge region of the IgG1 antibody heavy chain and the terminal end of the paired light chain, the H-L disulfide bond in this format is more stable and can be kept intact in the reducing condition during the drug conjugation to the antibody. This dramatically reduces the chances of obtaining ADCs which contains light chain drug conjugates.

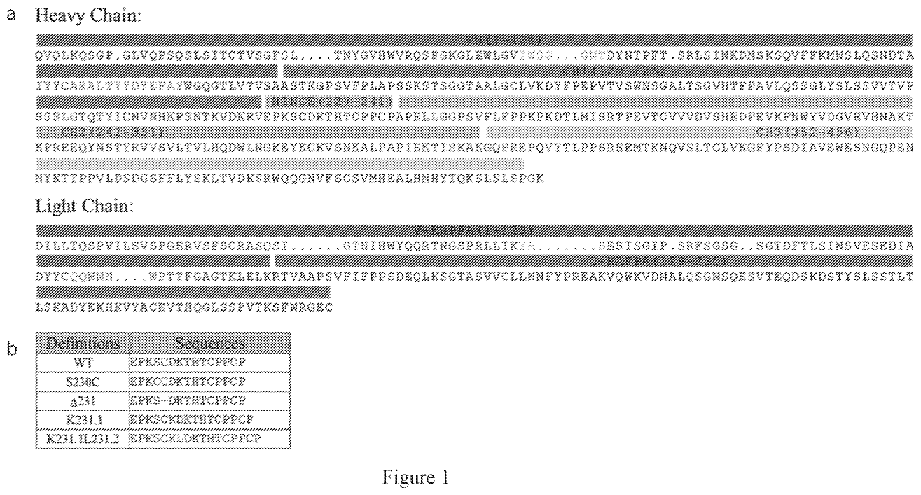

The IMGT numbering system for immunoglobulin superfamily is used herein to simplify the numbering scheme (schematic diagram in FIG. 1a), where the VH or VL domain each contain amino acid residues 1-128. Accordingly, amino acids in the CH1 domain are numbered as aa129-226; kappa domain as aa129-235; hinge region as aa227-241 (according to IgG1); CH2 as aa242-351, and CH3 as aa352-456 (see FIG. 1a). Based on this numbering scheme, the H-L inter-chain disulfide bond in wild-type IgG1(κ) would be formed between H(C231)-L(C235), while in IgG2(κ) (or IgG3(κ) or IgG4(κ)) it could be formed between H(C142)-L(C235). IgG1 mutant with heavy chain serein 230 changed to cysteine would be named IgG1(S230C), while with deletion of C231 would be named IgG1(Δ231). Insertion of a lysine after C231 would be named K231.1, and insertion of two amino acids, KL, after C231 would be named K231.1L231.2 (see FIG. 1b which shows a few examples of notations for mutations introduced in the hinge region of the IgG1). For example, the engineered antibodies of the present invention, antibody M35-1 and M45-1 include a modified hinge amino acid sequence comprising EPPKSCDKTHTVECPPCP (SEQ ID NO: 25), antibody M35-2 includes a modified hinge amino acid sequence comprising EPPKSDCKTKTVECPPCP (SEQ ID NO: 26). Note that antibody M35-1 and M45-1 include a human IgG1 CH1 with the mutation S142C, whereas antibody M35-2 contains a human IgG2 CH1.

In some embodiments, each of the heavy chains further comprises: a human Cl-1 domain located upstream of and connected to the hinge region, the CH1 domain comprising a cysteine at the position of 142 according to IMGT numbering scheme.

In another aspect, the present invention provides an isolated antibody, or an antigen-binding portion thereof, comprising: two heavy chains each comprising: (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; (b) a human CH1 domain located upstream of and connected to the hinge region, the CH1 domain comprising a cysteine at the position of 142 according to the IMGT numbering scheme, wherein the antibody specifically binds to human CD73 protein.

In some embodiments, the amino acid sequence of comprised in the hinge region is selected from the group consisting of SEQ ID NOs: 25-37, for example SEQ ID NO: 25 or SEQ ID NO: 26. In some embodiments, the antibody described herein can comprise two kappa light chains each paired with one of the heavy chains. The two heavy chains in the antibody can be identical. The two light chains in the antibody can be identical.

In some embodiments, the CH1 domain of the antibody has the same sequence as that of the CH1 domain of a native human IgG2, IgG3, or IgG4 subclass antibody, or the sequence of that of the CH1 domain of a native human IgG1 antibody with the mutation S142C.

In some embodiments, each of the heavy chains can further comprise an Fc domain of a native human IgG1, IgG2, IG3, IgG4 subclass antibody downstream of and connected to the hinge region, wherein the Fc domain optionally includes one or more substitutions, such as LALA mutations.

In some embodiments, each of the heavy chains comprises a variable domain comprising the amino acid sequence set forth in SEQ ID NO: 1 or SEQ ID NO: 3, and each of the light chains comprises a variable domain comprising the amino acid sequence set forth in SEQ ID NO: 2 or SEQ ID NO: 4.

In some embodiments, each of the heavy chains comprises an amino acid sequence set forth in one of SEQ ID NO: 17, SEQ ID NO: 19, and SEQ ID NO 21; and each of the heavy chains comprises an amino acid sequence set forth in one of SEQ ID NO: 18, SEQ ID NO: 20; and SEQ ID NO: 22.

In some embodiments, each of the heavy chains comprises an amino acid sequence set forth in one of SEQ ID NO: 17, and each of the light chains comprises an amino acid sequence set forth in one of SEQ ID NO: 18.

In some embodiments, each of the heavy chains comprises an amino acid sequence set forth in one of SEQ ID NO: 19, and each of the light chains comprises an amino acid sequence set forth in one of SEQ ID NO: 20.

In some embodiments, each of the heavy chains comprises an amino acid sequence set forth in one of SEQ ID NO: 21, and each of the light chains comprises an amino acid sequence set forth in one of SEQ ID NO: 22.

The term “isolated antibody” as used herein refers to an antibody that is substantially free of other antibodies having different antigenic specificities. An isolated antibody that specifically binds to an antigen is substantially free of antibodies that do not bind to that antigen.

The term “monoclonal antibody” as used herein refer to a preparation of a population of antibody molecules of substantially homogeneous molecular composition, wherein the individual antibodies in the population of the antibody molecules are identical except for possible naturally occurring mutations that may be present in miniscule amounts.

An antibody or molecule that “specifically binds to human CD73” refers to an antibody or polypeptide molecule that binds to human CD73 protein but does not substantially bind to proteins that are not human CD73 proteins.

DNA encoding an amino acid sequence variant of a starting polypeptide can prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may be constructed also by restriction fragment manipulation or by overlap extension PCR with synthetic oligonucleotides. Mutagenic primers encode the cysteine codon replacement(s). Standard mutagenesis techniques can be employed to generate DNA encoding such mutant engineered antibodies.

In yet a further aspect, the present disclosure provides a nucleic acid molecule encoding the antibody or antigen-binding portion thereof of any of the antibody described herein. A host cell (e.g., a CHO cell, a lymphocytic cell, a human embryonic kidney cell, or microorganisms, such as K coil and fungi, such as yeast) containing an expression vector containing the nucleic acid molecule, can be used to produce antibodies of the present disclosure, preferably monoclonal antibodies. In one embodiment, DNA encoding partial or full-length antibody of the present disclosure can be obtained by standard molecular biology techniques is inserted into one or more expression vectors such that the genes are operatively linked to transcriptional and translational regulatory sequences. The term “operatively linked” is intended to mean that an antibody gene is ligated into a vector such that transcriptional and translational control sequences within the vector serve their intended function of regulating the transcription and translation of the antibody gene. The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type I (Takebe et al., (1988) Mol. Cell. Biol. 8:466-472). The expression vector and expression control sequences are chosen to be compatible with the expression host cell used.

The antibody encoding DNA can be inserted into the expression vector. The recombinant expression vector can encode a signal peptide that facilitates secretion of the antibody chain from a host cell. The antibody encoding DNA can be cloned into the vector such that the signal peptide is linked in-frame to the amino terminus of the antibody encoding DNA. The signal peptide can be an immunoglobulin signal peptide or a heterologous signal peptide (i.e., a signal peptide from a non-immunoglobulin protein).

In a further aspect, an antibody-drug conjugate (ADC) or a pharmaceutically acceptable salt thereof, is provided. In some embodiments, the ADC comprises an antibody of the present disclosure as described herein, conjugated to a cytotoxic drug by a chemical linker.

In some embodiments, the cytotoxic drug can be selected from the group consisting of eribulin, monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, and nanoparticles. In other embodiments, the cytotoxic drug in the ADC is MMAE.

The chemical linker linking the antibody portion and the cytotoxic drug can be cleavable or non-cleavable. In some embodiments, the linker comprises a PEGn spacer where n is between 1 and 20 (i.e., having 1 to 20 repeat units (CH₂CH₂O)). In some embodiments, the chemical linker further comprises a linker segment connected to the PEGn spacer. In some embodiments, the chemical linker comprises a linker segment but does not comprise a PEGn spacer. In some embodiments, the chemical linker can include a segment that is selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGₙ-Val-Cit-PAB (n=1-20), Mal-amido-PEGn-Val-Cit-PAB (n=1-20), MC-Gly-Gly-Phe-Gly,Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate, or combinations thereof.

In the present disclosure, the pharmaceutically acceptable salts of the ADCs include acid addition salts of inorganic acids, carboxylic acids and sulfonic acids, for example, salts of the following acids: hydrochloric acid, hydrobromic acid, sulfuric acid, phosphoric acid, methanesulfonic acid, ethanesulfonic acid, benzenesulfonic acid, toluenesulfonic acid, naphthalene disulfonic acid, acetic acid, trifluoroacetic acid, propionic acid, lactic acid, tartaric acid, malic acid, citric acid, fumaric acid, maleic acid and benzoic acid.

The pharmaceutically acceptable salts of the antibody-drug conjugates of the present disclosure also include salts of conventional bases, for example alkali metal salts (e.g., sodium salts and potassium salts), alkaline earth metal salts (e.g., calcium salts and magnesium salts) and ammonium salts derived from ammonia or organic amines containing from 1 to 16 carbon atoms, in which the organic amines are, for example, ethylamine, diethylamine, triethylamine, ethyl diisopropylamine, monoethanolamine, diethanolamine, triethanolamine, dicyclohexylamine, dimethylaminoethanol, procaine, dibenzamide, N-methylpiperidine, N-methylmorpholine, arginine, lysine and 1,2-ethylenediamine.

It is understood that an ADC as used herein refers to a molecule that contains both a drug molecule and an antibody (or an antigen binding portion thereof) where the drug and the antibody (or the antigen binding portion thereof) is covalently connected by a linker. An “ADC preparation” herein refers to a collection or population of ADC molecules whose structure may differ due to possibly different attachment sites of the chemical linker to the antibody (or the antigen binding portion thereof). In some embodiments, the chemical linker is primarily or predominantly (e.g., ≥80%, ≥85%, ≥90%, ≥95%, or ≥98%) conjugated with cysteines on a heavy chain, resulting in an ADC preparation that is substantially devoid of light chain conjugation. In some embodiments, the chemical linker is conjugated with the antibody predominantly through the cysteines in the hinge region of the heavy chains of the antibody. And in certain embodiments, ADC molecules having drug to antibody ratio (DAR) of 2 accounts for at least 60%, at least 70%, at least 80%, at least 85%, or at least 90% of the total amount of ADC molecules.

In further aspect, the present disclosure provides a pharmaceutical composition comprising one or more antibodies, ADCs or the pharmaceutically acceptable salts thereof, of the present invention, together with a pharmaceutically acceptable carrier. As used herein, “pharmaceutically acceptable carrier” includes pharmaceutically acceptable carriers, excipients or stabilizers. These include but are not limited solvents, dispersion media, coatings, antibacterial and antifungal agents, isotonic and absorption delaying agents, surface active agents, thickening or emulsifying agents, solid binders, dispersion or suspension aids, solubilizers, colorants, flavoring agents, coatings, disintegrating agents, lubricants, sweeteners, preservatives, isotonic agents, and the like that are physiologically compatible. The selection of suitable carrier is within the knowledge of an artisan skilled in the art.

The composition may comprise one or more additional pharmaceutically active ingredients, such as another antibody, a drug, e.g., a cytotoxic or anti-tumor agent. The pharmaceutical compositions of the invention also can be administered in a combination therapy with, for example, another anti-cancer agent, another anti-inflammatory agent, etc.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the composition of the invention can be administered via a non-parenteral route, such as a topical, epidermal or mucosal route of administration, e.g., intranasally, orally, vaginally, rectally, sublingually or topically.

In a further aspect, the present invention provides a method of treating cancer in a human subject, comprising administering an effective amount of the pharmaceutical composition herein. The cancer can be a cancer associated with overexpression or upregulation of human CD73 protein. For example, the cancer can be selected from the group consisting of bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, glioma, melanoma, lymphoma, leukemia, myeloma, sarcoma, or virus-related cancer. The cancer may be a metastatic cancer, refractory cancer, or recurrent cancer.

In the administration of the composition to the subject, dosage regimens can be adjusted to provide the optimum desired response (e.g., a therapeutic response). Single bolus or divided doses can be administered based on the subject, the disease to be treated, etc. Dosage unit form as used herein refers to physically discrete units suited as unitary dosages for the subjects to be treated. Each unit contains a predetermined quantity of active ingredient calculated to produce the desired therapeutic effect in association with the required pharmaceutical carrier. Sustained release formulation can be used in which case less frequent administration is required.

For administration of an antibody or ADC pharmaceutical salts thereof of the present disclosure, the dosage may range from about 0.0001 to 100 mg/kg, and more usually 0.01 to 10 mg/kg, of the body weight of the subject. For example dosages can be 0.3 mg/kg body weight, 1 mg/kg body weight, 3 mg/kg body weight, 5 mg/kg body weight or 10 mg/kg body weight or within the range of 1-10 mg/kg. A suitable treatment regime can be once per week, once every two weeks, once every three weeks, once every four weeks, once a month, etc. Example dosage regimens for an anti-EGFR antibody of the invention can include 1 mg/kg body weight or 3 mg/kg body weight via intravenous administration.

A “therapeutically effective amount” or “effective amount” of an antibody or ADC or pharmaceutical salts thereof of the invention preferably results in a decrease in severity of disease symptoms, an increase in frequency and/or duration of disease symptom-free periods, prevention or reduction of likelihood of impairment or disability due to the disease affliction, or inhibition or delaying of the progression of disease. For example, for the treatment of tumor-bearing subjects, a “therapeutically effective amount” of an antibody composition may inhibits tumor growth by at least about 20%, more preferably by at least about 40%, even more preferably by at least about 60%, and still more preferably by at least about 80% relative to untreated subjects.

### Examples

### 1. Engineered M35-1, M45-1 and M35-2 Antibodies

Engineered antibodies for generating ADCs with high DAR2 ratios were designed with specifications shown in Table 1.

TABLE 1
Detailed features in the engineered antibodies.

| Clone ID of | | | | |
|---|---|---|---|---|
| engineered | | | | Hinge |
| antibodies | VH | VL | CH1 format | Sequences |
| M35-1 | SEQ ID NO: 1 | SEQ ID NO: 2 | IgG1S142C | SEQ ID NO: 25 |
| M35-2 | SEQ ID NO: 1 | SEQ ID NO: 2 | CH1 of IgG2 | SEQ ID NO: 26 |
| M45-1 | SEQ ID NO: 3 | SEQ ID NO: 4 | IgG1S142C | SEQ ID NO: 25 |

### 2. Expression and Purification of the M35-1, M35-2 and M45-1 Antibodies

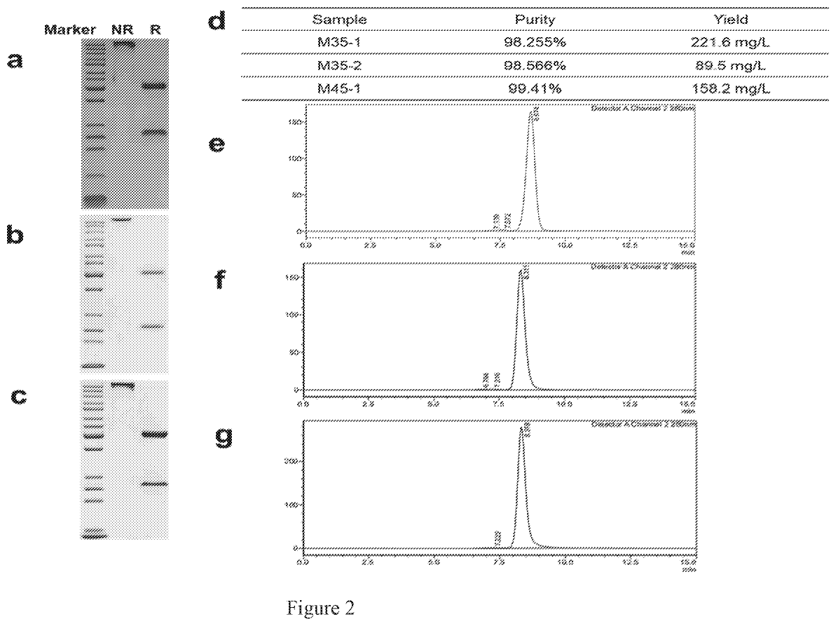

For the expression of the M35-1, M35-2 and M45-1 antibodies, codon optimization and gene synthesis were performed for better production in HEK293 cells. Full-length heavy chain and light chain DNA were each cloned into a separate pcDNA3 plasmid. HEK293 cell transient transfection of the paired plasmids and one-step Protein A purification was used to prepare enough proteins for testing. Antibodies made in this format expressed well with decent yield and could be purified in high purity with one step protein A purification process (FIG. 2).

### 3. Conjugation of M35-1, M35-2 and M45-1 Antibody to Generate ADCs

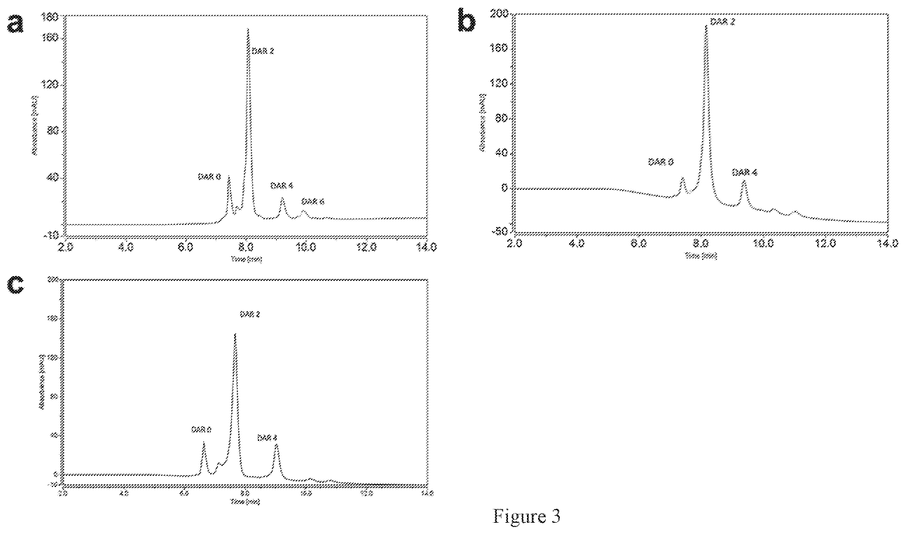

To generate the drug conjugates, TCEP was added to a PBS solution containing a purified antibody at neutral pH. Under mild reduction conditions (TCEP: mAb=1.1-2.4, neutral pH, room temperature for <240 min), inter-chain disulfide bonds of an antibodies were partially reduced. Drug-linker (MC-Val-Cit-PAB-MMAE) in DMA was added and allowed to react with antibody to obtain desired drug-to-antibody ratio (DAR). To characterize the ADCs, hydrophobic interaction chromatography (HIC) was performed for the evaluation of drug distribution and molar ratio of drug and antibody in ADCs, and the representative HIC chromatograms are presented in FIG. 3. Under these mild reduction/conjugation conditions, the ADCs made of all three engineered antibodies (M35-1, M35-2, and M45-1) were predominantly made of DAR2 ADC species, and the minor peaks are naked antibodies (DAR0) and DAR4 ADC species (FIGS. 3a, 3b, 3c).

4. Measurement of CD73 Binding Activities of Antibodies and their Corresponding ADCs

ELISA assay was used to evaluate and compare the CD73 binding capabilities of the antibodies and ADCs. Human CD73 proteins were coated onto 96-well plates and the plates were incubated at 4° C. overnight. Samples diluted in serial dilutions and S dilutions total. Diluted samples were then transferred to CD73 coated plates and incubated at room temperature for 1.5 h. Using HRP-labeled goat anti-human IgG Fc antibody (Sigma, A0170) as a detection agent and TMB for colorimetric reaction, the plates read at 450/650 nm for absorbance on Microplate Reader (Molecular Devices, SpectraMax 190) and data analysis was performed using a dose response curve format four parameters logistic model.

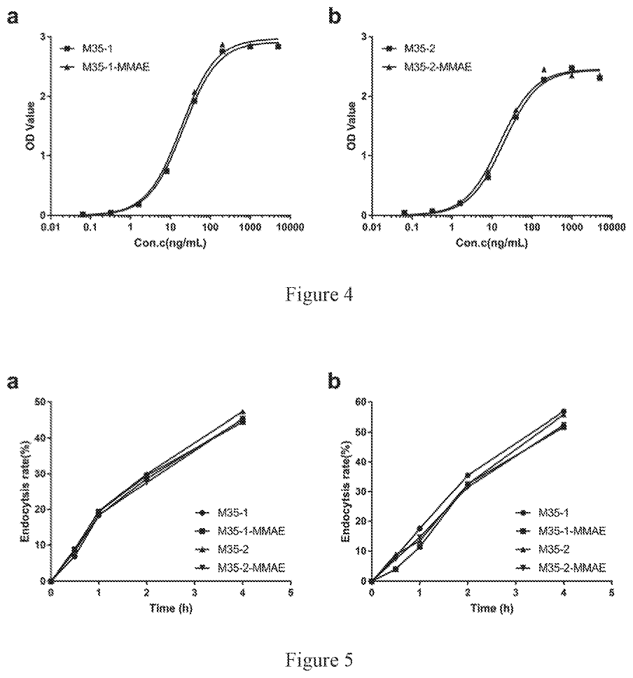

The results in FIG. 4 and Table 2 showed that the ADCs (M35-1-MMAE and M35-2-MM AE) made of M35-1 antibody or M35-2 antibody had similar binding activities to CD73 as compared with their corresponding naked antibodies, suggesting that payload conjugation did not affect target binding capability of ADCs.

TABLE 2
EC₅₀ (ng/mL) of CD73 binding activities with M35-1
and M35-2 antibodies, and their corresponding ADCs.

| M35-1 | M35-2 | M35-1-MMAE | M35-2-MMAE |
|---|---|---|---|
| 21.13 | 19.95 | 18.88 | 16.15 |

### 5. Measurement of Binding of Antibodies and ADCs to CD73-Expressing Cells

FACS assay was used to exam and compare the binding capabilities of the antibodies and ADCs to CD73 expressing cells. To perform the assay, target cells and samples were incubated at 4° C. for 1 h. After rinsing with PBS containing 0.5% BSA, samples and secondary antibody (goat pAb to human IgG (FITC) (Abcam, ab97224)) were incubated at 4° C. for 0.5 hrs. FACS was performed on low cytometer (B), Accuri C6 Plus) and mean fluorescence intensity (MFI) was reported. MFI of M35-1, M35-2 to multiple CD73 expressing cells was shown in Table 3. Four cell lines (NCI-H1975, NCI-H292, NCI-H441 and PC9) representing CD73 high- and low-expressing cells were used to compare binding capacities of different antibody constructs and ADCs. M35-1 and M35-2 antibodies, and their corresponding ADCs (M35-1-MMAE, M35-2-MMAE) had similar MFI levels on all tested cells (Table 4), suggesting that the CD73 binding activities were not affected by conjugation.

TABLE 3
CD73 expressing cells used in this work (CD73
expression levels were measured by FACS using
M35-1 antibody and MFIs were reported).

| Cell line (tumor type) | M35-1(MFI) |
|---|---|
| A431 (Skin) | 3.56 × 10⁴ |
| BT-474(Breast) | 3.88 × 10² |
| BXPC3 (pancreatic) | 9.26 × 10⁴ |
| LCLC-103H (lung) | 1.32 × 10⁵ |
| MDA-MB-468(Breast) | 1.17 × 10⁵ |
| NCI-N87 (Stomach) | 4.84 × 10⁴ |
| NUGC3 (Stomach) | 1.18 × 10⁵ |
| U87MG (Glioma) | 3.34 × 10⁵ |
| NCI-H1975 (lung) | 2.41 × 10⁵ |
| NCI-H292 (lung) | 7.13 × 10⁴ |
| NCI-H441 (lung) | 8.61 × 10⁴ |
| PC-9 (lung) | 7.72 × 10⁴ |

TABLE 4
MFI of the anti-CD73 antibodies and their corresponding
ADCs to CD73-expressing cells.

| Cell line | M35-1 | M35-1-MMAE | M35-2 | M35-2-MMAE |
|---|---|---|---|---|
| NCI-H1975 | 2.41 × 10⁵ | 2.79 × 10⁵ | 2.50 × 10⁵ | 2.75 × 10⁵ |
| NCI-H292 | 7.13 × 10⁴ | 7.66 × 10⁴ | 7.08 × 10⁴ | 7.54 × 10⁴ |
| NCI-H441 | 8.61 × 10⁴ | 9.33 × 10⁴ | 8.78 × 10⁴ | 9.10 × 10⁴ |
| PC-9 | 7.72 × 10⁴ | 8.11 × 10⁴ | 7.82 × 10⁴ | 7.84 × 10⁴ |

6. Internalization of Antibodies and ADCs into CD73 Expressing Cells

FACS based internalization assay was used to exam and compare the internalization of the naked antibodies and their corresponding ADCs into NCI-H1975 and NCI-H441, the CD73 high and low-expressing cells. The assay started with tubes of antibodies or ADCs (10000 ng/mL) mixed with target cells and chilled at 4° C. for 1 h, then the tubes were shifted to 37° C. Internalization of the antibody-target or ADC-target complex was measured up to 4 h. As shown in FIG. 5, both antibodies and ADCs could be internalized efficiently into NCI-H1975 and NCI-H441.

7. Cytotoxicity of ADCs to CD73 expressing Cells

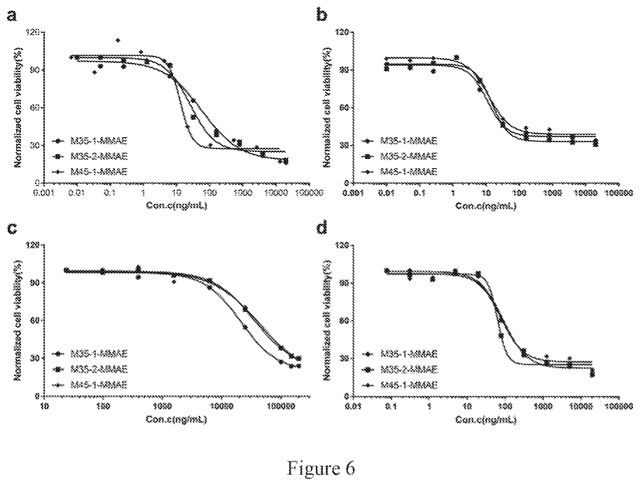

The cytotoxicity of the ADCs (M35-1-MAE, M35-2-MMAE or M45-1-MMAE) was evaluated and compared in in vitro cytotoxicity assay to multiple cell lines with different levels of CD73 expression. To perform the assay, target cells were seeded into a 96-well flat-bottom tissue culture plate at an optimized cell density for each cell line and incubated at 37° C., 5% CO₂ overnight (16-20 h). Serial dilutions of ADCs samples were transferred to cell plate and the assay plates were incubated for a defined period of time (3-7 days depend on cell lines) for optimal killing. Data analysis was performed using a dose response curve by four parameters logistic model. As shown in FIG. 6, M35-1-MMAE, M35-2-MMAE and M45-1-MMAE exerted similar cytotoxic potencies to CD73 high expressing U87MG cells, while M35-2-MMAE seemed more potent than M35-1-MMAE to NCI-H1975 cells. The potency differences seemed greater on CD73 low-expressing NCI-N87 and NCI-H292 cells.

### 8. In Vivo Efficacy of M35-1-MMAE and M35-2-MMAE Against Glioma Cancer Xenografts in Athymic Nude Mice.

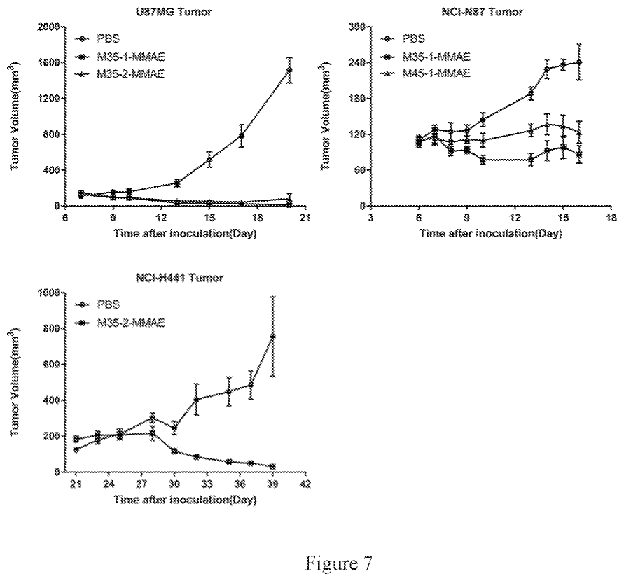

The antitumor potential of M35-1-MMAE and M35-2-MMAE was assessed in human U87 glioma, NCI-H1975 NSCLC and NCI-N87 gastric cancer xenografts model established in nude mice. Cancer cells were implanted in the back of athynic nude mice. Tumor-bearing mice received single i.v. treatment of either control (vehicle), M35-1, or M35-2 ADC. Tumor volume was measured at various time points after the treatment. The results shown in FIG. 7 demonstrated that treatment by anti-CD73 of high DAR2 ADCs not only could drastically suppress tumor growth in all tested models, they could cause tumor rejection in CD73 high-expressing tumor models (U87 and NCI-H1975 models).

### REFERENCES

[1] Saze Z, Schuler P J, et al., Blood, 2013; 122:9-18
[2] Jalkanen S, Salmi M. Arterioscler Thromb Vasc Biol, 2008; 28:18-26
[3] Willingham S B, Ho P Y, et al., Cancer Immunol Res. 2018, 6:1136-1149
[4] Lappas C M, Rieger J M, et al., J Immunol. 2005, 174:1073-80
[5] Ohta A, Gorelik E, Prasad S J, et al., Proc Natl Acad Sci USA. 2006; 103:13132-13127
[6] Waickman A T, Alme A, Senaldi L, et al., Cancer Immunol Immunother. 2012; 61:917-926
[7] Stagg J, Beavis P A, Divisekera U, et al., Cancer Res. 2012; 72:2190-2196
[8] Beavis P A, Milenkovski N, Henderson M A, et al., Cancer Immunol Res. 2015; 3:506-517
[9] Perrot 1, Michaud H A, Giraudon-Paoli M, et al., Cell Rep. 2019, 27:2411-2425
[10] Siu L L, Burris H, Le D T, et al., Cancer Res. 2018, 78
[11] Kjaergaard J, Hatfield S, Jones G, et al., J Immunol. 2018, 201:782-791
[12] Canon J, Rex K, Saiki A Y, et al., Nature 2019, 575:217-223.
[13] Jin R, Liu L, Xing Y, et al., Molecular Cancer Therapeutics, 2020

All patents and non-patent literature references described herein are incorporated by reference herein in their entireties.

While the invention has been described above in connection with one or more embodiments, it should be understood that the invention is not limited to those embodiments, and the description is intended to cover all alternatives, modifications, and equivalents, as may be included within the spirit and scope of the appended claims.

SEQUENCE LISTING

VH of M35-1 and M35-2:
QVQLVQSGAEVKKPGASVKVSCKTSGYTFTNYYIYWVRQAPGQRLEWMGWIYPGNL
NIKYNEKFKGRVTITADTSASTAYMELSSLRSEDTAVYYCARDDNYAWFAYWGQGT
LVTVSS (SEQ ID NO: 1)
VL of M35-1 and M35-2:
DIQMTQSPSSLSASVGDRVTITCKASQDVSTAVAWYQQKPGKAPKLLIYWTNTRHTG
VPSRFSGSGSGTDHTLTISSLQPEDFATYYCQQHYSTPFTFGQGTKLEIK (SEQ ID NO:
2)
VH of M45-1:
QVQLVQSGAEVKKPGASVKVSCKASGYTLTSYWMHWVRQAPGQGLEWMGEINPSQ
GRSNYNEKFKSRVTMTRDTSTSTVYMELSSLRSEDTAVYYCARRGVSGNYFDYWGQ
GTLVTVSS (SEQ ID NO: 3)
VL of M45-1:
DIQMTQSPSSLSASVGDRVTITCKASQDINTYLSWFQQKPGKSPKSLIYRSNILVSGVPS
RFSGSGSGQDYTLTISSLQPEDFATYYCLQYDEFPYTFGGGTKLEIK (SEQ ID NO: 4)
CDR1 of VH of M35-1 and M35-2:
NYYIY (SEQ ID NO: 5)
CDR2 of VH of M35-1 and M35-2:
WIYPGNLNIKYNEKFKG (SEQ ID NO: 6)
CDR3 of VH of M35-1 and M35-2:
DDNYAWFAY (SEQ ID NO: 7)
CDR1 of VL of M35-1 and M35-2:
KASQDVSTAVA (SEQ ID NO: 8)
CDR2 of VL of M35-1 and M35-2:
WINTRHT (SEQ ID NO: 9)
CDR3 of VH of M35-1 and M35-2:
DDNYAWFAY (SEQ ID NO: 10)
CDR1 of VH of M45-1:
SYWMH (SEQ ID NO: 11)
CDR2 of VH of M45-1:
EINPSQGRSNYNEKFKS (SEQ ID NO: 12)
CDR3 of VH of M45-1:
RGVSGNYFDY (SEQ ID NO: 13)
CDRI of VL of M45-1:
KASQDINTYLS (SEQ ID NO: 14)
CDR2 of VL of M45-1:
RSNILVSG (SEQ ID NO: 15)
CDR3 of VL of M45-1:
LQYDEFPYT (SEQ ID NO: 16)
Heavy Chain of M35-1:
QVQLVQSGAEVKKPGASVKVSCKTSGYTFTNYYIYWVRQAPGQRLEWMGWIYPGNL
NIKYNEKFKGRVTITADTSASTAYMELSSLRSEDTAVYYCARDDNYAWFAYWGQGT
LVTVSSASTKGPSVFPLAPCSKSTSGGTAALGCLVKDYFPEPVTVSWNSGALTSGVHT
FPAVLQSSGLYSLSSVVTVPSSSLGTQTYICNVNHKPSNTKVDKRVEPPKSCDKTHTVE
CPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK (SEQ ID NO:
17)
Light Chain of M35-1:
DIQMTQSPSSLSASVGDRVTITCKASQDVSTAVAWYQQKPGKAPKLLIYWTNTRHTG
VPSRFSGSGSGTDHTLTISSLQPEDFATYYCQQHYSTPFTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 18)
Heavy Chain of M35-2:
QVQLVQSGAEVKKPGASVKVSCKTSGYTFTNYYIYWVRQAPGQRLEWMGWIYPGNL
NIKYNEKFKGRVTITADTSASTAYMELSSLRSEDTAVYYCARDDNYAWFAYWGQGT
LVTVSSASTKGPSVFPLAPCSRSTSESTAALGCLVKDYFPEPVTVSWNSGALTSGVHTF
PAVLQSSGLYSLSSVVTVPSSNFGTQTYTCNVDHKPSNTKVDKTVEPPKSDCKTKTVEC
PPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK (SEQ ID NO:
19
Light Chain of M35-2:
DIQMTQSPSSLSASVGDRVTITCKASQDVSTAVAWYQQKPGKAPKLLIYWTNTRHTG
VPSRFSGSGSGTDHTLTISSLQPEDFATYYCQQHYSTPFTFGQGTKLEIKRTVAAPSVFI
FPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC (SEQ ID NO: 20)
Heavy Chain of M45-1:
QVQLVQSGAEVKKPGASVKVSCKASGYTLTSYWMHWVRQAPGQGLEWMGEINPSQ
GRSNYNEKFKSRVTMTRDTSTSTVYMELSSLRSEDTAVYYCARRGVSGNYFDYWGQ
GTLVTVSSASTKGPSVFPLAPCSKSTSGGTAALGCLVKDYFPEPVTVSWNSGALTSGV
HTFPAVLQSSGLYSLSSVVTVPSSSLGTQTYICNVNHKPSNTKVDKKVEPPKSCDKTHT
VECPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK (SEQ ID
NO: 21)
Light Chain of M45-1:
DIQMTQSPSSLSASVGDRVTITCKASQDINTYLSWFQQKPGKSPKSLIYRSNILVSGVPS
RFSGSGSGQDYTLTISSLQPEDFATYYCLQYDEFPYTFGGGTKLEIKRTVAAPSVFIFPP
SDEQLKSGTASVVCLLNNFYPREAKVQWKVDNALQSGNSQESVTEQDSKDSTYSLSS
TLTLSKADYEKHKVYACEVTHQGLSSPVTKSENRGEC (SEQ ID NO: 22)
CH1 of M35-1 and M45-1 (in the format of IgG1S142C):
ASTKGPSVFPLAPCSKSTSGGTAALGCLVKDYFPEPVTVSWNSGALTSGVHTFPAVLQ
SSGLYSLSSVVTVPSSSLGTQTYICNVNHKPSNTKVDKRV (SEQ ID NO: 23)
CH1 of M35-2 (in the format of IgG2):
ASTKGPSVFPLAPCSRSTSESTAALGCLVKDYFPEPVTVSWNSGALTSGVHTFPAVLQS
SGLYSLSSVVTVPSSNFGTQTYTCNVDHKPSNTKVDKTV (SEQ ID NO: 24)
Hinge Sequence of Heavy Chain of M35-1 and M45-1:
EPPKSCDKTHTVECPPCP (SEQ ID NO: 25)
Hinge Sequence of Heavy Chain of M35-2:
EPPKSDCKTKTVECPPCP (SEQ ID NO: 26)
Hinge Sequence of Heavy Chain of Other Examples:
CKTHTCPPCP (SEQ ID NO: 27)
DKTCHTCPPCP (SEQ ID NO: 28)
ERKSCVECPPCP (SEQ ID NO: 29)
EPKSDCKTHTCPPCP (SEQ ID NO: 30)
EPKSDKCTHTCPPCP (SEQ ID NO: 31)
EPKSDCKTHTVECPPCP (SEQ ID NO: 32)
EPKSDCKTVECPPCP (SEQ ID NO: 33)
EPKSDKCTHTVECPPCP (SEQ ID NO: 34)
EPPPKSCDKTHTVECPPCP (SEQ ID NO: 35)
EPPPPKSCDKTHTVECPPCP (SEQ ID NO: 36)
EPPKSDCKTKTVECPPCP (SEQ ID NO: 37)

What is claimed is:

1. An isolated antibody or an antigen-binding portion thereof, comprising:
two heavy chains each comprising:
(a1) a heavy chain hinge region comprising the amino acid sequence set forth in any of SEQ ID NOs: 25-37; and
(a2) a heavy chain variable domain comprising a CDR1 region, a CDR2 region, and a CDR3 region comprising the amino acid sequences of SEQ ID NO: 5, SEQ ID NO: 6, and SEQ ID NO: 7, respectively, and
two light chains each comprising:
(a3) a light chain variable domain comprising a CDR1 region, a CDR2 region, and a CDR3 region comprising the amino acid sequences of SEQ ID NO: 8, SEQ ID NO: 9, and SEQ ID NO: 10, respectively.

2. An isolated antibody or an antigen-binding portion thereof, comprising:
two heavy chains each comprising:
(b1) a heavy chain hinge region comprising the amino acid sequence set forth in any of SEQ ID NOs: 25-26;
(b2) a heavy chain variable domain comprising a CDR1 region, a CDR2 region, and a CDR3 region comprising the amino acid sequences of SEQ ID NO: 11, SEQ ID NO: 12, and SEQ ID NO: 13, respectively, and
two light chains each comprising:
(b3) a light chain variable domain comprising a CDR1 region, a CDR2 region, and a CDR3 region comprising the amino acid sequences of SEQ ID NO: 14, SEQ ID NO: 15, and SEQ ID NO: 16, respectively.

3. The antibody or the antigen-binding portion thereof, of any of claims 1-2, wherein the antibody specifically binds to human CD73 protein.

4. The antibody, or the antigen-binding portion thereof, of any of claims 1-3, wherein each of the heavy chains further comprises: a human CH1 domain located upstream of and connected to the hinge region, the CH1 domain comprising a cysteine at the position of 142 according to IMGT numbering scheme.

5. An isolated antibody, or an antigen-binding portion thereof, comprising:
two heavy chains each comprising:
(a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue;
(b) a human CH1 domain located upstream of and connected to the hinge region, the CH1 domain comprising a cysteine at the position of 142 according to the IMGT numbering scheme;
wherein the antibody specifically binds to human CD73 protein.

6. The antibody or the antigen-binding portion thereof, of claim 5, wherein the amino acid sequence of comprised in the hinge region is selected from the group consisting of SEQ ID NOs: 25-37.

7. The antibody or the antigen-binding portion thereof, of any of claims 5-6, wherein the amino acid sequence comprised in the heavy chain hinge region is SEQ ID NO: 25.

8. The antibody or the antigen-binding portion thereof, of any of claims 5-6, wherein the amino acid sequence comprised in the heavy chain hinge region is SEQ ID NO: 26.

9. The antibody, or the antigen-binding portion thereof, of any of claims 5-8, further comprising two kappa light chains each paired with one of the heavy chains.

10. The antibody, or the antigen-binding portion thereof, of any of claims 4-9, wherein the CH1 domain of the antibody has the same sequence as that of the CH1 domain of a native human IgG2, IgG3, or IgG4 subclass antibody.

11. The antibody, or the antigen-binding portion thereof, of any of claims 4-9, wherein the CH1 domain of the antibody has the sequence of that of the CH1 domain of a native human IgG1 antibody with the mutation S142C.

12. The antibody, or the antigen-binding portion thereof, of any of claims 4-9, wherein each of the heavy chains further comprises a Fe domain of a native human IgG1, IgG2, IgG3, IgG4 subclass antibody downstream of and connected to the hinge region, wherein the Fc domain optionally includes one or more substitutions.

13. The antibody, or the antigen-binding portion thereof, of any of the foregoing claims, wherein each of the heavy chains comprises a variable domain comprising the amino acid sequence set forth in SEQ ID NO: 1 or SEQ ID NO: 3, and wherein each of the light chains comprises a variable domain comprising the amino acid sequence set forth in SEQ ID NO: 2 or SEQ ID NO: 4.

14. The antibody, or the antigen-binding portion thereof, of any of the foregoing claims, wherein each of the heavy chains comprises an amino acid sequence set forth in one of SEQ ID NO: 17, SEQ ID NO: 19, and SEQ ID NO: 21; and wherein each of the heavy chains comprises an amino acid sequence set forth in one of SEQ ID NO: 18, SEQ ID NO: 20; and SEQ ID NO: 22.

15. The antibody, or the antigen-binding portion thereof, of any of the foregoing claims, wherein:
(a) each of the heavy chains comprises an amino acid sequence set forth in one of SEQ ID NO: 17; and each of the light chains comprises an amino acid sequence set forth in one of SEQ ID NO: 18; or
(b) each of the heavy chains comprises an amino acid sequence set forth in one of SEQ ID NO: 19; and each of the light chains comprises an amino acid sequence set forth in one of SEQ ID NO: 20; or
(c) each of the heavy chains comprises an amino acid sequence set forth in one of SEQ ID NO: 21; and each of the light chains comprises an amino acid sequence set forth in one of SEQ ID NO: 22.

16. An antibody-drug conjugate (ADC) or a pharmaceutically acceptable salt thereof, comprising:
the antibody of any of the claims 1-15 conjugated to a cytotoxic drug by a chemical linker.

17. The ADC or a pharmaceutically acceptable salt thereof, of claim 16, wherein the cytotoxic drug is selected from the group consisting of eribulin, monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, and nanoparticles.

18. The ADC or a pharmaceutically acceptable salt thereof, of any of claims 16 or 17, wherein the chemical linker comprises a portion that is selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGₙ-Val-Cit-PAB (n=1-20), Mal-amido-PEGn-Val-Cit-PAB (n=1-20), MC-Gly-Gly-Phe-Gly, Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate.

19. The ADC or a pharmaceutically acceptable salt thereof, of any of claims 15 or 16, wherein the cytotoxic drug is MMAE.

20. A pharmaceutical composition comprising: an isolated antibody or an antigen binding portion thereof of any of claims 1-15, or an ADC of pharmaceutically acceptable salt thereof, of claims 16-19, and a pharmaceutical acceptable carrier.

21. A method of treating cancer in a human subject, comprising administering an effective amount of the pharmaceutical composition of claim 20.

22. The method of claim 21, wherein the cancer is associated with overexpression of human CD73 protein.

23. The method of claim 21, wherein the cancer is selected from the group consisting of bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, glioma, melanoma, lymphoma, leukemia, myeloma, sarcoma, or virus-related cancer.
